(19) United States
(12) Patent Application Publication
(10) Pub. No.: US 2018/0256093 A1
(43) Pub. Date: Sep. 13, 2018

(54) DIAGNOSTIC APPARATUS

(71) Applicants: UNIVERSITE CLAUDE BERNARD LYON 1, Villeurbanne (FR); CENTRE NATIONAL DE LA RECHERCHE SCIENTIFIQUE (CNRS), Paris (FR); INSTITUT NATIONAL DES SCIENCES APPLIQUEES DE LYON, Villeurbanne (FR); CPE LYON FORMATION CONTINUE ET RECHERCHE pouvant etre precedee ou suivie par CPE LYON FCR LYON, Villeurbanne (FR); ECOLE CENTRALE DE LYON, Ecully (FR); HOSPICES CIVILS DE LYON, Lyon (FR)

(72) Inventors: Olivier ROBIN, Lyon (FR); Claudine GEHIN, Venissieux (FR); Bertrand MASSOT, Lyon (FR)

(21) Appl. No.: 15/756,567

(22) PCT Filed: Aug. 26, 2016
(86) PCT No.: PCT/EP2016/070238

§ 371 Date: Feb. 28, 2018

(30) Foreign Application Priority Data
Aug. 31, 2015 (FR) 1558064

(51) Int. Cl. A61B 5/00; A61C 7/36

(52) U.S. Cl. CPC A61B 5/4557; A61F 2005/563; A61C 7/36; A61B 5/682

(57) ABSTRACT

The invention relates to a diagnostic apparatus comprising a mounting (20) and a measurement circuit (15) attached to the mounting, the mounting comprising a gutter (12) having a shape which substantially complements that of a dental arch, the measurement circuit comprising at least one force sensor (24) arranged at the bottom of said gutter, a clock (28), a memory (30), a control module (32) capable of recording in said memory the force measurements taken by said at least one force sensor and the corresponding times read by said clock, and a battery (36) for supplying the measurement circuit with electrical energy, the mounting comprising at least two layers encapsulating said measurement circuit, the apparatus being characterised in that the measurement circuit comprises a sheet substrate (26), said substrate comprising first and second portions connected by at least one bridge (42) having a width (l) of less than 2 mm.

### TECHNICAL FIELD

The invention relates to diagnostic apparatus designed for detecting bruxism and evaluating its nature.

### PRIOR ART

Treatment of bruxism is generally performed without prior precise diagnosis. In fact, to date polysomnography is considered as the sole reliable solution for such diagnosis. But this technique is delicate and costly to carry out. Also, it requires a hospital stay. There is therefore a risk that treatment is not properly adapted.

Also, WO2012175634 discloses detection apparatus including a gutter provided with contactors. To detect any bruxism, the gutter is fixed to the upper arch of the patient. The contactors are disposed so as to close under the effect of a clenching force exerted by the teeth and likely to correspond to bruxism.

The detection apparatus described in WO2012/175634 is not very reliable. In fact, the force needed to close a contactor is predetermined whereas the level of the force corresponding to bruxism depends on the patient. Bruxism can be suspected for example when the chewing forces of a patient are two or three times greater than normal chewing forces. These normal chewing forces vary from one individual to the other, especially as a function of gender.

Above all, the detection apparatus described in WO2012/175634 does not enable such diagnosis.

There is therefore a need for apparatus to resolve the above problems at least partially. An aim of the invention is to respond to this need.

### SUMMARY OF THE INVENTION

The invention relates to diagnostic apparatus including a support and a measurement circuit fixed on the support,

the support including a gutter having a form substantially complementary to that of a dental arch,
the measurement circuit including at least one force sensor disposed at the base of said gutter, a clock, a memory, a control module capable of recording in said memory the force measurements measured by said at least one force sensor and the corresponding instants measured by said clock, and a battery for supplying the measurement circuit with electrical power.

According to the invention, the support includes at least two layers encapsulating said measurement circuit.

As will be seen in greater detail in the rest of the description, diagnostic apparatus according to the invention can be compact and can therefore be worn comfortably, especially outside the hospital. The encapsulation also sealingly insulates the measurement circuit.

Diagnostic apparatus according to the invention can also have one or more of the following optional features:
the force sensor is a piezoresistive sensor;
the apparatus includes an internal communication module, the internal communication module and the battery being disposed on either side of a median longitudinal plane of the apparatus;
the measurement circuit includes first and second parts connected by at least one bridge having a width (l) of less than 2 mm;
said bridge presents a loop, preferably more than 2, more than 5, preferably more than 10 loops.

The invention also relates to a conformed measurement circuit to be encapsulated between two layers of a support of apparatus according to the invention.

Preferably, a measurement circuit according to the invention includes first and second parts connected by at least one bridge, preferably by a bridge having a loop. Advantageously, a measurement circuit according to the invention is easily deformable, and therefore adaptable to several apparatuses according to the invention intended for different patients.

In particular, the flexibility of the measurement circuit is preferably adapted so that apparatus according to the invention can be manufactured according to a method as per the invention described hereinbelow.

The invention also relates to a method for manufacturing diagnostic apparatus according to the invention. This method includes the following steps:
a) manufacture of a measurement circuit;
b) sandwiching of the measurement circuit between two plastically deformable sheets, so as to form a sandwich structure;
c) plastic deformation of the sandwich structure so as to form a gutter having the general form of a dental arch of a patient and preferably form an arch having the general form of the palate of said patient and fastening of the two sheets to each other so as to encapsulate the measurement circuit between said sheets.

Surprisingly, the inventors have noted that such a method, simple to execute, sealingly insulates the measurement circuit from its environment. Also, the two sheets can be welded to each other without the need for intermediary resin. Any risk associated with any possible toxicity from the resin is therefore advantageously removed.

Finally, such a method enables relative precise positioning of the sheets and of the measurement circuit during step b). Such positioning is more difficult with a resin which adheres instantly to the pieces with which it comes into contact.

The invention also relates to a method for calibrating the gain of a sensor of apparatus according to the invention, including the following steps:
i. determination of a maximum measuring range and minimum and maximum thresholds, said thresholds being preferably determined as a function of said maximum measuring range;
ii. when the measurement passes above the maximum threshold or below the minimum threshold, increase or decrease, respectively, of the maximum measuring range, and updating of the maximum measuring range as a consequence.

The invention also relates to a kit including apparatus according to the invention or made according to a method as per the invention, and a base, the apparatus and the base including an internal communication module and an external communication module, respectively, said internal and external communication modules being capable of communicating between each other by means of electromagnetic waves of frequency greater than 50 kHz and less than 30 MHz, to transfer data recorded in said memory to said external communication module.

The base preferably has a keying pin shaped to ensure positioning of said apparatus on the base in a recharge position in which the internal communication module is substantially facing the external communication module.

### BRIEF DESCRIPTION OF THE FIGURES

Other features and advantages of the invention will emerge more clearly from the following detailed description and on examination of the appended drawing, in which

FIG. 1 illustrates, in perspective, an example of apparatus according to the invention;

FIG. 2 illustrates the apparatus of FIG. 1, according to the sectional plane A;

FIG. 3 illustrates, in perspective, the measurement circuit of the apparatus of FIG. 1;

FIG. 4 illustrates an example of a bridge of the substrate of FIG. 3;

FIG. 5 illustrates a measurement circuit, prior to insertion between the two thermodeformable sheets; and

FIG. 6 illustrates the method for manufacturing the diagnostic apparatus.

### DEFINITIONS

A “force sensor” is a sensor for measuring a force, in a measuring range. A force sensor is different to a force detector which, as for a contactor, detects only if a force threshold has been exceeded.

“Patient” means any person for whom apparatus according to the invention is implemented to diagnose bruxism, whether or not this person is ill, or this person is undergoing treatment or not.

Unless indicated otherwise, “including a”, “having a”, or “comprising a” means “including at least one”.

### DESCRIPTION OF A DETAILED EMBODIMENT

### Apparatus

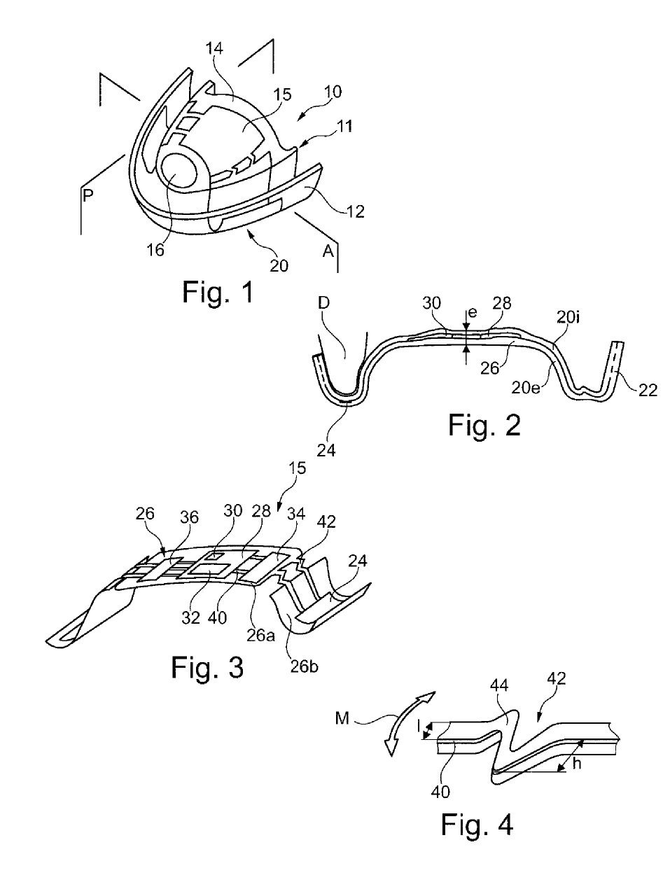

As shown in FIG. 1, apparatus 10 according to the invention includes a support 11 constituted by a gutter 12 and an arch 14, and a measurement circuit 15 fixed to the support, preferably disposed on the arch 14.

The gutter 12 has the general form of a “U” with two branches. It is shaped to receive teeth D of the upper arch of a patient. Preferably, the gutter is personalised and has the general form of the imprint of the teeth of the patient. Comfort and precision of measurements are considerably improved.

The arch 14 preferably has the form of the upper wall of the mouth of the patient, or “palate”. The arch 14 is preferably connected to the two branches of the gutter. Preferably, it is connected at least to the free ends of the branches of the gutter.

The gutter 12 and/or the arch 14 can be continuous or by comparison have one or more recesses 16. In particular, the arch can be lightened by eliminating those parts of the arch which do not encapsulate the measurement circuit 15. The gutter can be also lightened, for example by omitting that part of the gutter which would cover the upper surfaces of incisors and/or canines when in the service position.

In a preferred embodiment, the arch forms a veil which extends continuously between the two branches of the gutter, the junction line between the arch and the gutter preferably being continuous and preferably extending between the two ends of the branches of the gutter, extending along a “U-shaped” curve.

As shown in FIG. 2, the support 11 includes an internal layer 20i, intended to be in contact with the teeth D and the palate, and an external layer 20e oriented towards the oral cavity. The internal and external layers encapsulate the measurement circuit 15, i.e., there is a bonding area 22 between the two internal and external layers which sealingly encircles the measurement circuit 15. The circuit 15 is hermetically insulated from the exterior.

The internal and external layers are fixed to each other, by any means, for example by adhesion, preferably by welding, according to the bonding areas 22. More preferably, the internal and external layers are fixed to each other in all the areas in which they do not sandwich the measurement circuit 15.

Preferably, the internal and/or external layers are made of transparent material, and/or thermoformable material, and/or biocompatible material. Preferably, the internal and external layers are made of the same material. Preferably, the material constituting the internal and/or external layers is selected from thermoformable plastic materials, preferably PETG (Polyethylene terephthalate glycol-modified).

Preferably, the internal layer and/or the external layer have a maximum thickness less than 2 mm, preferably less than 1 mm, and/or greater than 0.5 mm. Preferably, the thickness of the internal and/or external layers is substantially constant. Advantageously, the thickness of the apparatus is reduced, and the apparatus is therefore comfortable.

An example of a measurement circuit 15 is shown in FIG. 3. The measurement circuit 15 illustrated includes one, preferably several, force sensors 24, and a substrate 26 on which electronic components are fixed, preferably all the components electronic. Preferably, the substrate supports at least the sensor(s) 24, a clock 28, a memory 30 for storing data, a control module 32 capable of recording in the memory 30 the force measurements taken by the sensor(s) 24 and timestamping them by means of time data supplied by the clock 28, and a battery 36 feeding the electronic components with electric power needing electric power to function (clock, control module and sensors especially). Preferably, the measurement circuit also includes an internal communication module 34.

Preferably, the components of the measurement circuit are distributed so that the centre of gravity of the measurement circuit is substantially in the median longitudinal plane P of the apparatus, or “sagittal plane”. In particular, the internal communication module 34 and the battery 36 are preferably disposed on either side of the median longitudinal plane P, improving comfort.

A sensor 24 is preferably a piezoresistive sensor, i.e., a component whereof the electrical resistance depends on the compression force which is exerted on it. Preferably, at least one sensor 24 is disposed so as to be clenched between at least two posterior teeth when the patient clenches his teeth. Advantageously, a piezoresistive sensor is particularly thin, improving the comfort of the patient.

The sensor 24 is preferably fixed to the substrate 26.

In a preferred embodiment, the sensor 24 is set as a function of the patient for whom the apparatus is intended.

Advantageously, the measuring precision is improved.

The number of force sensors 24 can be greater than 1, greater than 2, greater than 3, greater than 4, greater than 5 or greater than 6.

Preferably, the measurement circuit is configured so that the gain of the sensor is modified, in real time, as a function of the measurements taken. Preferably, adaptation is made according to the following steps:
i. determination of a maximum measuring range and minimum and maximum thresholds, said thresholds being preferably determined as a function of said maximum measuring range;
ii. when the measurement passes above the maximum threshold or below the minimum threshold, increase or decrease, respectively, of the maximum measuring range, and updating of the maximum measuring range as a consequence.

For example, the maximum measuring range can be initially 1 V, i.e., the sensor supplies for example voltage between 0 and 1 V as a function of the force exerted on it. The value of 1 V can for example correspond to a force of 80 Newton. With this setting, any force greater than 80 Newton will be transcribed by the sensor by a voltage 1 V, falsifying the measurement.

To avoid this problem, at step ii., maximum and minimum thresholds are determined, for example 75% and 25% of the maximum measuring range, respectively, i.e., 0.75 V and 0.25 V.

When the measured force leads to a signal over the maximum threshold of 0.75 V, the maximum measuring range is modified and rises, for example, from 1 V to 2 V. The maximum and minimum thresholds are recalculated. Preferably, these thresholds are a fraction of the maximum measuring range, for example illustrate 25% and 75% of the maximum measuring range, respectively.

Updating of the maximum and minimum thresholds results in values of 1.5 V and 0.5 V. Modification of the maximum measuring range illustrates modification of the gain of the sensor and allows the latter to continue to measure forces by avoiding the level described hereinabove, corresponding to saturation. The sensor can continue its measurements. If the measured force results in voltage above the new maximum threshold, the maximum measuring range is again increased and the maximum and minimum thresholds are reupdated.

If the voltage drops below the minimum threshold, equaling 0.5 V, the maximum measuring range is reduced, for example to return to 1 V and the threshold values are reupdated as a consequence. The decrease in the maximum measuring range improves precision for low forces.

The dynamic adaptation of the gain advantageously allows very good measuring quality, irrespective of the patient using the device. Preferably, the material constituting the internal and external layers is rigid, i.e., does not deform under the effect of normal clenching of teeth, at least in the part where the sensors 24 are disposed, so as to effectively transmit the clenching forces exerted by the teeth of the patient to the sensors 24.

The substrate 26 can especially comprise any sheet material usually used for manufacture of electronic circuits, for example a polyimide sheet. The substrate 26 preferably has a thickness of less than 1 mm, preferably less than 0.5 mm, preferably less than 0.4 mm, preferably, has a thickness less than 0.3 mm, preferably less than 0.2 mm, preferably less than 0.1 mm.

Conventionally, conductive tracks 40, for example made of copper, are printed onto the substrate 26 so as to electrically connect the different components.

Preferably, the substrate 26 includes first and second parts 26a and 26b, respectively, connected together by one or more bridges 42.

Preferably, at least one, preferably each one, of the parts 26a and 26b connected by one or more bridges has a surface greater than 1 cm², 2 cm², 3 cm², or even 5 cm², 8 cm², or 10 cm².

Preferably, the substrate includes a central part 26a configured to extend exclusively over the palate of the patient, and/or one, or preferably two, lateral parts 26b. The central part is preferably configured so as to extend substantially exclusively over the palate and/or the lateral part(s) is (are) preferably configured to extend substantially exclusively in the gutter.

The bridge(s) 42 is (are) thus in areas subject to high-level flexions and tensions.

The bridges 42 are shaped so as to enable, before the measurement circuit is sandwiched, displacement of the first part 20a relative to the second part 20b. The minimum width l of a bridge, i.e., the width of the bridge at the place where the bridge is the least wide, is preferably greater than 0.2 mm and/or less than 1 mm. Preferably, the width of a bridge is substantially constant.

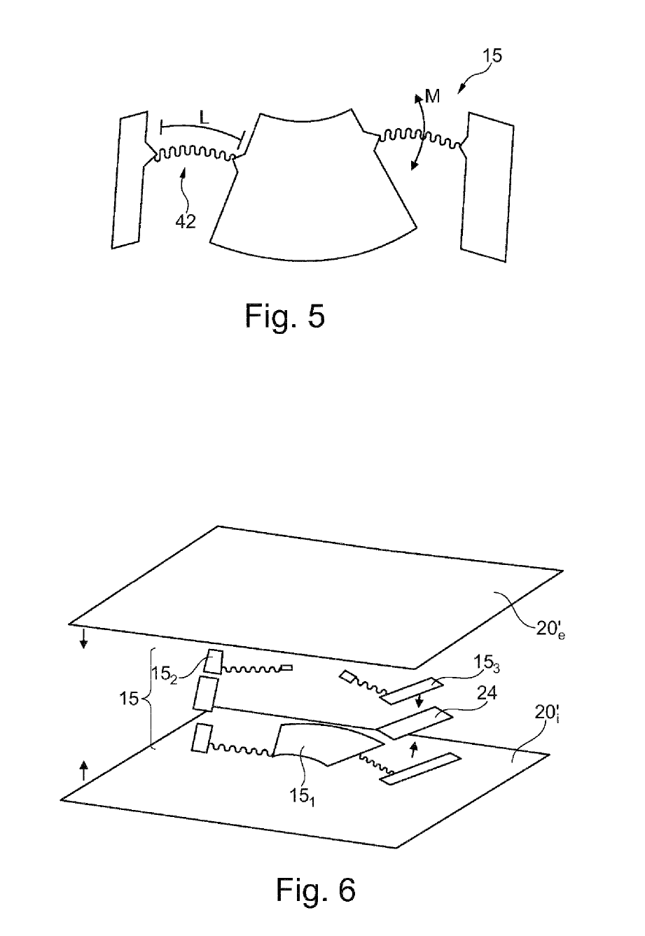

As shown in FIG. 5, the length L of a bridge is preferably greater than 5 mm, preferably greater than 8 mm, or even greater than 10 mm, and/or less than 20 mm, preferably less than 15 mm. The unfolded length of a bridge is preferably greater than 10 mm and/or less than 30 mm. The bridge is preferably configured so that its length L can be increased by more than 5%, more than 10%, more than 13%, more than 17%, or even more than 20%.

Preferably, a bridge 42 has at least one loop 44 for easy longitudinal and/or transversal deformation of the bridge 42. The transversal deformation of the bridge 42 is illustrated by arrow M in FIG. 4. The height h of a loop 44 is preferably greater than 0.5 mm and/or less than 2 mm, preferably less than 1.5 mm, preferably less than 1.0 mm, preferably less than 0.8 mm. The form of a loop 44 is not limiting. In particular, it can be rounded (in the form of Omega in FIG. 5), pointed (FIG. 4), or meandering, for example.

Preferably, a bridge includes more than 2, more than 5, preferably more than 10, or even more than 15 loops (see FIG. 5).

As shown in FIG. 4, a bridge 42 can carry a track 40. In an embodiment, the track 40 which extends over a bridge 42 extends over the entire width of the bridge 42, i.e., covers it fully.

As will be evident in more detail hereinbelow, making a deformable substrate, and in particular including bridges, advantageously manufactures apparatuses for different patients from a standard measurement circuit.

Electronic components of the measurement circuit, in particular the clock 28, and/or the memory 30 and/or the control module 32, can be integrated into a “chip” (integrated circuit).

The control module 32 is programmed so as to timestamp and record the measurements taken by the sensor 24.

The control module controls the measurements by the sensor 24 continuously or not, preferably at a frequency greater than 1 Hz, preferably greater than 2 Hz and/or preferably less than 10 Hz. It associates each measurement with a date and a time (timestamping) corresponding to the instant when the measurement was taken. The recordings are stored in the memory 30.

The reading and/or writing speed of the memory 30 is preferably greater than 100 kb its/sec.

Preferably, the control module 32 is programmed so as to control the internal communication module 34. Preferably, the internal communication module 34 includes a transmitter and, preferably, a receiver, including a winding adapted to communicate inductively with an external communication module, not shown. Preferably, the frequency of the electromagnetic waves emitted by the internal communication module 34 is greater than 50 kHz, preferably greater than 100 kHz and/or less than 30 MHz, preferably less than 200 kHz.

In a preferred embodiment, the internal communication module 34 is passive, i.e., it makes use of the energy of the received electromagnetic wave to reply. Preferably, this response consists of modification to this wave, which is then sent back.

Communication is carried out preferably by means of a base on which the apparatus according to the invention is placed. Preferably, the base includes a keying pin ensuring adapted positioning of the apparatus on the base, in a position so-called “recharge position”.

Preferably, the base is partly in a form complementary to the form of the support 11. For example, the base can have a protrusion cooperating with the apparatus to ensure precise positioning of said apparatus on the base. For example, the base can include beading of material corresponding substantially to the form of the gutter 12. Preferably, in the recharge position, the internal communication module 34 is substantially facing the external communication module, integrated into the base, favouring inductive coupling and therefore the exchange of data and/or power.

Preferably, the electronic components of the measurement circuit are powered by means of energy stored in the battery 36. Preferably, the battery 36 is rechargeable, preferably contactless, preferably by induction, preferably by means of electromagnetic waves of a frequency greater than 50 kHz.

The features of apparatus according to the invention reduce its weight and improve comfort. The weight of apparatus according to the invention is preferably under 50 g. More preferably, in a section according to a median transversal plane, as for plane A of FIG. 1, the maximum thickness e of the apparatus 10 is less than 5 mm, less than 3 mm, as shown in FIG. 2.

Preferably, a module for processing data unloaded from the apparatus according to the invention, not shown, determines:
the intensity of the force exerted by the teeth during clenching phases, i.e., when the sensor 24 is compressed between the jaws of the patient, and/or
the duration of the clenching phases, and/or
the frequency of the clenching phases, and/or
the moments of the day when the clenching phases occur.

Preferably, the processing module is programmed to perform diagnosis of the state of the patient and preferably produce a presentation report of this diagnosis.

### Manufacturing Method

Apparatus according to the invention can be made according to steps a) to c) described hereinabove.

In step a), the manufacture of the measurement circuit 15 can be performed by any known method for making an electronic circuit. Preferably, electrically conductive tracks 40 are printed onto the substrate 26, then the electronic components are connected electrically to said tracks.

The substrate is preferably flexible.

The tracks are drawn out as a function of the electrical connections to be set up. In conventional terms, on one of its faces or on both its faces the substrate includes a layer of copper which is selectively removed to leave the tracks.

The tracks are also traced to ensure flexibility of the circuit adapted to the later steps. In particular, one or more bridges 42, having preferably one or more loops, can be provided in areas of the substrate intended to be deformed in step c).

As shown in FIG. 6, the measurement circuit can include several parts 15₁, 15₂ and 15₃, which can be manufactured as indicated previously.

As shown in FIG. 6, the sensors 24 can be sandwiched between the part 15₁ on the one hand and the parts 15₂ and 15₃ on the other. Other embodiments are possible, of course.

Preferably, in the areas of the bridges 42, the substrate is cut out on each side of each bridge 42, as in FIGS. 4 and 5.

In step b), the support of the measurement circuit 15, preferably substantially planar, is disposed sandwiched between two sheets 20i′ and 20e′, made of thermoformable material, which will form the internal 20i and external 20e layers, respectively. Preferably, the sheets are substantially planar and overflow from all sides around the measurement circuit 15.

Preferably, the sandwich includes only the measurement circuit and the two sheets. Preferably, no binder, and in particular no resin, is sandwiched between the two sheets. Advantageously, the position of the measurement circuit between the two sheets and/or the relative position of the two sheets can be modified as needed to where the preferred arrangement is achieved.

Preferably, they are cut out together. Preferably, after cutting out, the overflow of the assembly comprising the two sheets beyond the measurement circuit is greater than 2 mm, or even 5 mm.

Preferably, the edges of the apparatus are then softened to limit the risk of injury.

In step c), the sandwich structure formed by the two sheets and the measurement circuit is deformed so as to produce the gutter 12 and the arch 14. Preferably, the gutter 12 and the arch 14 forms a single-piece assembly, and preferably results from deformation of a single sandwich structure.

Preferably, the gutter, and preferably the arch, are obtained by application and deformation of the sandwich structure on a model, for example made of plaster, of the upper arch of the patient. The form of the support 11 is then fully adapted to the patient for whom the apparatus is intended. Both comfort and efficacy of measuring are advantageously improved.

Preferably, the sheets are made of thermoformable material. In step c), the sandwich structure is brought to a temperature enabling deformation of said sheets. After the preferred form is achieved, the temperature is returned to ambient temperature, resulting in hardening of said sheets.

Preferably, during step c), the sheets stick to each other in the bonding areas 22 in which they are in contact with each other, preferably by welding, i.e., local fusion of said sheets. This local fusion is particularly advantageous as it ensures very good sealing of the bond, which properly protects the measurement circuit.

After testing of many resins, the inventors discovered that this embodiment, technically simpler, was also the most effective.

Preferably, the temperature applied at step c) is greater than 150° C. and/or less than 300° C. Advantageously, such temperatures do not degrade the functioning of the electronic components of the measurement circuit.

The heating period is preferably greater than 20 s and/or less than 50 s.

After fastening of both sheets to each other according to the bonding areas 22, the overflow of the sheets all around the measurement circuit 15 insulates the measurement circuit from the exterior.

As shown in FIG. 3, the bridges 42 enable deformation of the measurement circuit. Advantageously, the same measurement circuit 15 can serve to manufacture apparatuses designed for different patients. Also, the bridges 42 improve the robustness of the apparatus, and therefore prolong its service life.

After hardening of the sheets, the form of the apparatus can be refined, for example by deburring, chamfering, cutting, sanding or machining, to produce a form as ergonomic as possible.

### Operation

Apparatus according to the invention is advantageously very light and thin, such that it can be worn without stress by the patient.

During a bruxism phase, the patient abnormally clenches his teeth and therefore compresses the sensor 24 between his two jaws. The electrical resistance of the sensor 24 evolves as a consequence. At regular intervals, or even ongoing, the control module evaluates the resistance of the sensor 24. It records this resistance, or likewise a corresponding force, in the memory 30, by associating it with the date and time of the measurement.

Measurement by intermittence advantageously limits electrical consumption. A measurement frequency of 1 or 2 Hz is considered satisfactory.

The data stored during test phases calibrate the sensor 24 to heighten measuring precision. The data stored during diagnosis phases serve to determine diagnosis.

After a measuring phase, which can last for over a day, more than two days, over a week, or even more than two weeks, the apparatus 10 is placed on a base, the gutter being preferably placed on a beading of corresponding shape, preferably so that the winding of the internal communication module 34 is facing a corresponding winding of the external communication module 50 of the base. Inductive coupling between these windings lets the external communication module 50 send an electromagnetic wave, preferably at a frequency of around 150 kHz. The winding of the internal communication module 34 can convert some of this energy into electrical energy and store it in the battery 36 to power the electronic components of the apparatus during the following measuring phase. The internal communication module 34 can also send back a wave to the external communication module 50 modified so as to transmit data recorded in the memory 30.

These data are transmitted to a processing module capable presenting them, for example on a screen or in the form of a report. Preferably, the processing module analyses the data and determines aggregated values useful for setting up diagnosis. Preferably still, the processing module sets up diagnosis.

As will now become evident, the invention provides a solution for diagnosing bruxism simply and comfortably for patients, and follows its evolution in the long term.

Of course, the invention is not limited to the embodiments described and illustrated, provided for illustrative purposes only.

1. Diagnostic apparatus including a support and a measurement circuit fixed to the support,
the support including a gutter having a form substantially complementary to that of a dental arch,
the measurement circuit including at least one force sensor disposed at the base of said gutter, a clock, a memory, a control module capable of recording in said memory the force measurements measured by said at least one force sensor and the corresponding instants measured by said clock, and a battery for supplying the measurement circuit with electrical power, the support including at least two layers encapsulating said measurement circuit,
the apparatus being characterised in that the measurement circuit includes a substrate made of a sheet, said substrate including first and second parts connected by at least one bridge having a width of less than 2 mm.

2. The apparatus according to claim 1, wherein said bridge has a loop.

3. The apparatus according to claim 1, wherein said bridge supports a conductive track.

4. The apparatus according to claim 1, wherein the support includes an arch having the form of the palate of the patient.

5. The apparatus according to claim 1, wherein the first and second parts extend substantially exclusively over the arch and over the gutter, respectively.

6. The apparatus according to claim 1, wherein said at least one force sensor is a piezoresistive sensor.

7. The apparatus according to claim 6, including an internal communication module, the internal communication module and the battery being disposed on either side of a median longitudinal plane of the apparatus.

8. The apparatus according to claim 7, wherein the components of the measurement circuit are distributed so that the centre of gravity of the measurement circuit is substantially in the median longitudinal plane of the apparatus.

9. The apparatus according claim 1, wherein said layers are made of thermoformable material.

10. The apparatus according to claim 1, the measurement circuit being configured so that the gain of the sensor is modified, in real time, as a function of the measurements taken, according to the following steps:
determination of a maximum measuring range and minimum and maximum thresholds, said thresholds being preferably determined as a function of said maximum measuring range;
when the measurement passes above the maximum threshold or below the minimum threshold, increase or decrease, respectively, of the maximum measuring range, and updating of the maximum measuring range as a consequence.

11. A method for manufacturing a diagnostic apparatus according to claim 1, said method including the following steps:
a) manufacture of the measurement circuit;
b) sandwiching of the measurement circuit between two plastically deformable sheets, so as to form a sandwich structure;
c) plastic deformation of the sandwich structure so as to form a gutter having the general form of a dental arch of a patient and an arch having the general form of the palate of said patient and fastening of the two sheets to each other so as to encapsulate the measurement circuit between said sheets.

12. A kit comprising:
a diagnostic apparatus manufactured according to the method of claim 11,
a base,
the apparatus and the base including an internal communication module and an external communication module, respectively, said internal and external communication modules for communicating between each other by means of electromagnetic waves of frequency greater than 50 kHz and less than 30 MHz, to transfer data recorded in said memory to said external communication module.

13. The kit according to the claim 12, wherein the base has a keying pin shaped to ensure positioning of said apparatus on the base in a recharge position in which the internal communication module is substantially facing the external communication module.
